(19) United States
(12) Patent Application Publication
(10) Pub. No.: US 2026/0355347 A1
(43) Pub. Date: Nov. 12, 2026

(54) ROUTE OF ADMINISTRATION

(71) Applicant: UCB BIOPHARMA SRL, Brussels (BE)

(72) Inventors: Patrick Mark Downey, Brussels (BE); Toshal Rohit Patel, Brussels (BE); Amulya Nidhi Shrivastava, Brussels (BE)

(21) Appl. No.: 19/124,740

(22) PCT Filed: Oct. 30, 2023
(86) PCT No.: PCT/EP2023/080220

§ 371 Date: Apr. 25, 2025

(30) Foreign Application Priority Data
Oct. 31, 2022 (GB) 2216168.1

(51) Int. Cl. A61K 48/00; A61K 38/17; C12N 7/00

(52) U.S. Cl. CPC A61K 48/0016; A61K 38/1709; A61K 48/0075; C12N 7/00; C12N 2750/14132; C12N 2750/14143; C12N 2830/36; C12N 2830/42; C12N 2830/50

(57) ABSTRACT

The invention relates to the treatment of a neurodegenerative diseases, via intra-putaminal route of administration, with recombinant adeno-associated viral (rAAV) particles comprising an AAV true type (AAVTT) capsid and a heterologous nucleic acid packaged therein.

### FIELD OF INVENTION

The invention relates to the field of route of administration of recombinant adeno-associated viral (rAAV) particles, namely rAAV particles comprising an AAV true type (AAVTT) capsid and a heterologous nucleic acid packaged therein, for use in the treatment of a neurodegenerative diseases, as well as pharmaceutical compositions comprising said particles.

### BACKGROUND OF THE INVENTION

Neurodegenerative diseases affect adult and paediatric patients. They often have progressive and relentless evolution which leads to the degeneration of the structure and function of the central and peripheral nervous systems. A common feature for most of these diseases is the severe impairment of combination of cognitive, motor and sensory functions, leading to loss of quality of life and ultimately death of the patients. Neurodegenerative diseases vary in etiology, prevalence, diagnosis, and management, leading to heterogenous patient populations.

Treatments for neurodegenerative diseases are confined to symptoms treatment and patients still lack disease-modifying therapies capable of treating the disease, by reversing, halting or slowing the prognosis, removing the symptoms or even curing the disease.

Gene therapy has the strong potential for treating neurodegenerative diseases, and a large number of preclinical and clinical studies are currently in place for addressing this need.

Adeno-associated virus (AAV) has been the predominant choice for central or peripheral nervous system-focused clinical trials. A number of factors make AAV an ideal gene delivery vehicle for neurodegenerative diseases. Vectors based on AAV are particularly promising gene delivery vehicles in large part because they exhibit low immunogenicity, have a low risk of insertional mutagenesis and can mediate long-term gene expression in both dividing and non-dividing cells, a necessity for neurodegenerative diseases (Ojala D. S. et al, 2015).

Systemic routes of administration are not particularly suited for AAV-based gene therapy for the treatment of neurodegenerative disease. To date, intracranial administration has been a commonly employed route for AAV-based gene therapy delivery to the brain (Wood et al, 2022). This approach circumvents the biological transport barrier (the so called “blood-brain barrier”) and further reduces the risk of vector neutralisation by circulating antibodies. Intracranial administration does have significant drawbacks. Poor vector spread limits transgene expression to the vicinity of the administration site, a major shortcoming for diseases that affect multiple regions of the central nervous system. For example, AAV2 has particularly strong neuronal tropism (Bartlett J. S. et al., 1998) and has been favoured in clinical trials for its established safety records. Yet, other serotypes such as AAV1, AAV5 or AAV9, have been shown to have high efficiency in transducing neurons (Mandel R. J. et al. 2004; Sawamoto K. et al, 2018; Wang D. et al., 2019).

Amongst neurodegenerative diseases, frontotemporal dementia (FTD) is a fatal neurodegenerative disease that typically presents with deficits in executive function, behaviour, speech or comprehension. These symptoms are associated with a characteristic pattern of brain atrophy affecting the frontal and temporal cortices and subcortical areas such as the thalamus and the hippocampus (Whitwell J. L., et al., 2011; Liscic R. M., 2017). Patients universally exhibit a progressive course, with an average survival of 8 years from symptom onset (Coyle-Gilchrist I. T. et al., 2016). In 5-10% of FTD patients, pathogenic loss-of-function mutations can be identified in the granulin (GRN) gene encoding progranulin (PGRN), a ubiquitous lysosomal protein (Rohrer J D. et al., 2009). GRN mutation presents with clinical features of other neurodegenerative diseases, such as progressive supranuclear palsy, corticobasal syndrome, Parkinson's disease, dementia with Lewy bodies, Alzheimer's disease (Le Ber I., et al. 2008), mucopolysaccharidosis (MPS), neuronal ceroid lipofuscinosis (NCL), a group of progressive neurodegenerative disorders characterized by intracellular accumulation of ceroid lipopigments (Kamate M. et al., 2019) and amyotrophic lateral sclerosis (ALS), optionally with dementia (ALS-D) (Irwin D. et al., 2009). AAV-based gene therapies to correct GRN mutations and thus, provide patients sufficient level of PGRN, have been recently described (WO22034130; WO2019070894; WO2019070894). In particular, intra-ventricular delivery has been described as a preferred route of administration for GRN AAV-based gene therapies (WO2017151884). Most recently, and intra-cisterna magna (ICM) AAV1- or AAV9-based gene therapies (WO22046988; ClinicalTrial.gov NCT04747431 and NCT04408625) have been also reported (WO22046988).

Rodent and non-human primate surrogates are still irreplaceable models for developing gene therapy treatment for human uses. Rodent models remain important for exploring pathways and disease mechanisms as well as identifying potential treatment targets. However, the resemblance (in some cases the nearly identical networking) of primate neurological structures to those of humans render the use of primate models the only meaningful approach to investigate which brain area should be successfully targeted with a specific viral vector to establish the most effective route of administration for neurodegenerative diseases for human uses (Wozar F. et al., 2022; Pignataro D. et al., 2018; FDA draft guidance 2022).

Thus, there remains a need for identifying the most effective route of administration for disease-modifying therapies (such as AAV-based gene therapies) for neurodegenerative diseases (including adult-onset neurodegenerative diseases) that affect large central nervous system regions such as FTD. In particular, there remains the need to identify the most effective route of administration for the treatment of GRN mutations with an AAVtt-based gene therapy.

### SUMMARY OF THE INVENTION

In a first aspect, the invention provides a method of treating a neurodegenerative disease in a primate by administering to the primate a therapeutic amount of recombinant adeno-associated viral (rAAV) particles, wherein the rAAV particles comprise: a) an AAV true type (AAVTT) capsid comprising or consisting of SEQ ID NO: 1 and b) a heterologous nucleic acid packaged therein; wherein the heterologous nucleic acid comprises: i) a 5′ inverted terminal repeat (ITR) or a fragment thereof and a 3′ ITR or a fragment thereof; ii) a transgene; and iii) one or more regulatory sequences that direct expression of said transgene; wherein the treatment comprises the administration of said rAAV particle to the putamen of said primate.

In a second aspect, the invention relates to a method of delivering to the central nervous system of a primate a therapeutic amount of recombinant adeno-associated viral (rAAV) particles, wherein the rAAV particles comprise: a) an AAV true type capsid comprising or consisting of SEQ ID NO: 1 and b) a heterologous nucleic acid packaged therein; wherein the heterologous nucleic acid comprises: i) a 5′ inverted terminal repeat (ITR) or a fragment thereof and a 3′ ITR or a fragment thereof; ii) a transgene; and iii) one or more regulatory sequences that direct expression of said transgene; wherein the method comprises administering the rAAV particles to the putamen of said primate.

In a third aspect, the invention provides recombinant adeno-associated viral (rAAV) particles for use in the treatment of a neurodegenerative disease in a primate, wherein the rAAV particles comprise: a) an AAV true type (AAVTT) capsid comprising or consisting of SEQ ID NO: 1 and b) a heterologous nucleic acid packaged therein; wherein the heterologous nucleic acid comprises: i) a 5′ inverted terminal repeat (ITR) or a fragment thereof and a 3′ ITR or a fragment thereof; ii) a transgene; and iii) one or more regulatory sequences that direct expression of said transgene; wherein the rAAV particles are administered to the putamen of said primate.

In a fourth aspect, the invention relates to recombinant adeno-associated viral (rAAV) particles for use in the delivery of a heterologous nucleic acid to the central nervous system of a primate, wherein the rAAV particles comprise: a) an AAV true type (AAVTT) capsid comprising or consisting of SEQ ID NO: 1 and b) the heterologous nucleic acid to be delivered, packaged therein; wherein the heterologous nucleic acid comprises: i) a 5′ inverted terminal repeat (ITR) or a fragment thereof and a 3′ ITR or a fragment thereof; ii) a transgene; and iii) one or more regulatory sequences that direct expression of said transgene; wherein the rAAV particles are administered to the putamen of said primate.

In a fifth aspect, the invention describes a pharmaceutical composition comprising recombinant adeno-associated viral (rAAV) particles and one or more carriers and/or excipients, wherein the rAAV particles comprise: a) an AAV true type capsid comprising or consisting of SEQ ID NO: 1 and b) a heterologous nucleic acid packaged therein; wherein the heterologous nucleic acid comprises: i. a 5′ inverted terminal repeat (ITR) or a fragment thereof and a 3′ ITR or a fragment thereof; ii. a transgene; and iii. one or more regulatory sequences that direct expression of said transgene; wherein the pharmaceutical composition is administered to the putamen of a primate in need thereof. Said composition is preferably for use in the treatment of a neurodegenerative disease in a primate.

In an embodiment of all these aspects, the transgene encodes a secreted lysosomal protein, preferably human progranulin (PGRN), an active fragment thereof and/or an active variant thereof. Additional aspects and embodiments of the invention will be disclosed herein below.

### DEFINITIONS

Unless defined otherwise, all technical and scientific terms used herein have the same meaning as is commonly understood by one of skill in art to which the subject matter herein belongs. As used in the specification and claims, the following definitions are supplied to facilitate the understanding of the present invention.
The term “and/or” used in a phrase such as “A and/or B” herein is intended to include “A and B”, “A or B”, “A”, and “B”.
The singular forms “a”, “an”, and “the” include plural referents unless the content clearly dictates otherwise. Thus, for example, reference to “a recombinant AAV particle” includes “recombinant AAV particles”, and the like.
The term “comprising” does not exclude other elements. For the purposes of the present disclosure, the term “consisting of” is considered to be a preferred embodiment of the term “comprising of”.
The term “viral particle” relates to an infectious and typically replication-defective virus particle comprising (i) at least a portion of a viral vector packaged within (i) a capsid and optionally, (iii) a lipidic envelope surrounding the capsid. The term “Viral particle” includes recombinant Adeno Associated Viral particle.
The terms “recombinant Adeno Associated Viral particle”, “recombinant AAV particle”, “rAAV particle”, or alternatively simply “particle”, encompasse viral particle comprising AAV capsids including the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

The heterologous nucleic acid comprises the transgene to be expressed in the target cell, at least one or more regulator sequences driving the expression of the transgene as well as inverted terminal repeat sequences (ITRs) from a viral vector. Except for the ITRs, the heterologous nucleic acid is mainly made of non-AAV genome sequences.
The term “AAV true type”, “AAVT”, AAV-TT” or “AAVtt” relates to a capsid as defined in WO2015121501 and Tordo J. et al., 2018, both incorporated herein by reference, and comprising or consisting of SEQ ID NO: 1. A “r” in front of any of these terms stands for recombinant.
The terms “inverted terminal repeat sequences”, “inverted terminal repeat” or “ITR” are sequences located at the 5′ and 3′ ends of the heterologous nucleic acid which allows the transgene and the one or more regulatory sequences located between the ITRs to be packaged within the rAAV capsid. Although typically full length ITRs from the same source as the one providing the rep function are used, as an alternative, ITRs from a different AAV source can be used, as well as truncated ITRs as long as they are still functional.
The term “regulatory sequence” refers to one or more sequences that direct and/or are involved in the expression of a gene (herein of the transgene). Typically said one or more regulatory sequences are selected to drive, assist and/or control the expression of the transgene in the target tissue, e.g. central nervous system (CNS).
The term “transgene” refers to the nucleic acid sequence (typically encoding a protein) to be expressed in a primate once administered to the primate via the rAAV particles according to the invention, wherein said sequence which is not an AAV-derived sequence. It is typically of the same origin as the primate to be treated with the rAAV particle. The term “a transgene” should be construed as comprising one or more transgenes.
The term “heterologous nucleic acid” refers to the nucleic acid sequence packaged inside the rAAV capsid which forms a viral particle. Such a nucleic acid sequence contains AAV inverted terminal repeat sequences (ITRs). In the examples herein, a heterologous nucleic acid contains, at a minimum, from 5′ to 3′, an AAV 5′ ITR, (a) sequence(s) encoding a transgene (i.e. a gene different from the gene encoding viral proteins), and an AAV 3′ ITR.
The terms “therapeutic amount” or “therapeutical effective amount” typically refer to the amount or the dose of a compound that is sufficient to exhibit a positive pharmacologic and/or physiologic effect on a disease and therefore to treat a disease, upon administration to a primate.
The terms “treatment”, “treating” and the like, refer to obtaining a desired pharmacologic and/or physiologic effect. The effect may be prophylactic in terms of completely or partially preventing a disease or symptom thereof to appear or to worthen and/or may be therapeutic in terms of a partial or complete cure for a disease and/or adverse effect attributable to the disease. Treatment thus covers any treatment of a disease in a primate, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

### BRIEF DESCRIPTION OF THE DRAWINGS

FIG. 1: Coronal sections of NHP hemisphere stained for transgene expression (herein PRGN). A) and B) are intra-putaminal administrations of vehicle and rAAV-TT, respectively. C) shows the staining for intra-ventricular administration of rAVV-TT.

### DETAILED DESCRIPTION OF THE INVENTION

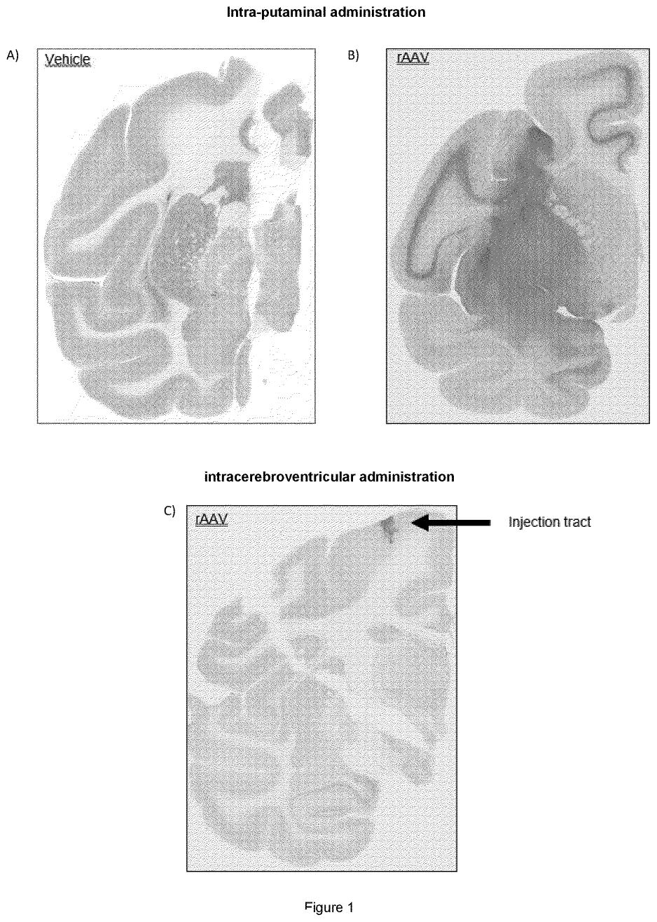

The invention is based on the surprising finding that recombinant adeno-associated virus (rAAV) particles comprising AAV true type (AAVTT) capsids were able, when administered to the putamen, to spread from the putamen to the central nervous system (CNS) and to express the progranulin-encoding transgene comprised in said particle all across the CNS (in particular to cortical and subcortical regions) of the primate in need of treatment. The inventors have also unexpectedly found that the administration to the putamen of rAAV particles according to the invention is superior to an intracerebroventricular (ICV) administration of the same rAAV particle in said primate (FIG. 1).

Therefore, the present invention provides a method of treating a neurodegenerative disease in a primate by administering to the primate a therapeutic amount of a recombinant adeno-associated viral (rAAV) particle, wherein the rAAV particle comprises:
a) an AAV true type (AAVTT) capsid comprising or consisting of SEQ ID NO: 1 and
b) a heterologous nucleic acid packaged therein; wherein the heterologous nucleic acid comprises:
   i) a 5′ inverted terminal repeat (ITR) or a fragment thereof and a 3′ ITR or a fragment thereof;
   ii) a transgene; and
   iii) one or more regulatory sequences that direct expression of said transgene;

wherein the treatment comprises the administration of said rAAV particle to the putamen of said primate, and wherein the transgene encodes a secreted lysosomal protein, preferably human progranulin (PGRN), an active fragment thereof and/or an active variant thereof.

The second object of the present invention is a method of delivering to the central nervous system (CNS) of a primate a therapeutic amount of a recombinant adeno-associated viral (rAAV) particle, wherein the rAAV particle comprises:
a) an AAV true type capsid comprising or consisting of SEQ ID NO: 1 and
b) a heterologous nucleic acid packaged therein; wherein the heterologous nucleic acid comprises:
   i) a 5′ inverted terminal repeat (ITR) or a fragment thereof and a 3′ ITR or a fragment thereof;
   ii) a transgene; and
   iii) one or more regulatory sequences that direct expression of said transgene; wherein the method comprises administering the rAAV particle to the putamen of said primate, and wherein the transgene encodes a secreted lysosomal protein, preferably human progranulin (PGRN), an active fragment thereof and/or an active variant thereof.

The third object of the present invention is a recombinant adeno-associated viral (rAAV) particle for use in the treatment of a neurodegenerative disease in a primate, wherein the rAAV particle comprises:
a) an AAV true type (AAVTT) capsid comprising or consisting of SEQ ID NO: 1 and
b) a heterologous nucleic acid packaged therein, wherein the heterologous nucleic acid comprises:
   i) a 5′ inverted terminal repeat (ITR) or a fragment thereof and a 3′ ITR or a fragment thereof;
   ii) a transgene; and
   iii) one or more regulatory sequences that direct expression of said transgene;

wherein the rAAV particle is administered to the putamen of said primate, and wherein the transgene encodes a secreted lysosomal protein, preferably human progranulin (PGRN), an active fragment thereof and/or an active variant thereof.

The fourth object of the present invention is a recombinant adeno-associated viral (rAAV) particle for use in the delivery of a heterologous nucleic acid to the central nervous system of a primate, wherein the rAAV particle comprises:
a) an AAV true type (AAVTT) capsid comprising or consisting of SEQ ID NO: 1 and
b) a heterologous nucleic acid packaged therein, wherein the heterologous nucleic acid comprises:
   i) a 5′ inverted terminal repeat (ITR) or a fragment thereof and a 3′ ITR or a fragment thereof;
   ii) a transgene; and
   iii) one or more regulatory sequences that direct expression of said transgene;

wherein the rAAV particle is administered to the putamen of said primate, and wherein the transgene encodes a secreted lysosomal protein, preferably human progranulin (PGRN), an active fragment thereof and/or an active variant thereof.

The fifth object of the present invention is a pharmaceutical composition comprising a recombinant adeno-associated viral (rAAV) particle and one or more carriers and/or excipients, wherein the rAAV particle comprises:
a) an AAV true type capsid comprising or consisting of SEQ ID NO: 1 and
b) a heterologous nucleic acid packaged therein; wherein the heterologous nucleic acid comprises:
   i. a 5′ inverted terminal repeat (ITR) or a fragment thereof and a 3′ ITR or a fragment thereof;
   ii. a transgene; and
   iii. one or more regulatory sequences that direct expression of said transgene;

wherein the pharmaceutical composition is administered to the putamen of a primate in need thereof, and wherein the transgene encodes a secreted lysosomal protein, preferably human progranulin (PGRN), an active fragment thereof and/or an active variant thereof. Said composition is preferably for use in the treatment of a neurodegenerative disease in a primate.

In the context of the invention as a whole, once administered to the putamen, the particles are able to spread within the central nervous system (CNS) so that they will be delivered all across its areas as shown by the expression of the transgene throughout the CNS.

In the context of the invention as a whole, the 5′ ITR comprises or consists of: i) SEQ ID NO: 2, or a sequence having at least 95%, at least 96%, at least 97%, at least 98% or at least 99% sequence identity thereto; or ii) SEQ ID NO: 5 or a sequence having at least 95%, at least 96%, at least 97%, at least 98% or at least 99% sequence identity thereto; and the 3′ ITR comprises or consists of: i) SEQ ID NO: 3, or a sequence having at least 95%, at least 96%, at least 97%, at least 98% or at least 99% sequence identity thereto, or ii) SEQ ID NO: 6 or a sequence having at least 95%, at least 96%, at least 97%, at least 98% or at least 99% sequence identity thereto. In a non-limiting example, the 5′ITR comprises or consists of SEQ ID NO: 2 and the 3′ITR comprises or consists of SEQ ID NO: 3. In another non-limiting example, the 5′ITR comprises or consists of SEQ ID NO: 5 and the 3′ITR comprises or consists of SEQ ID NO: 6.

In the context of the invention as a whole, the heterologous nucleic acid comprises a transgene encoding a secreted lysosomal protein |(including pre and/or pro-form of such secreted proteins). The secreted lysosomal proteins preferably human progranulin (PGRN), an active fragment thereof and/or an active variant thereof, wherein PGRN has preferably an amino acid sequence comprising SEQ ID NO: 4 or a sequence having at least 95%, at least 96%, at least 97%, at least 98% or at least 99% sequence identity thereto. A non-limiting example of a nucleic sequence encoding PGRN comprises SEQ ID NO: 7 or a sequence having at least 95%, at least 96%, at least 97%, at least 98% or at least 99% sequence identity thereto.

The heterologous nucleic acid according to the present invention comprises one or more regulatory sequences that direct, assist and/or control the expression of said transgene in the primate to be treated by gene therapy with the rAAV particle as described herein. In the context of the invention as a whole, the one or more regulatory sequences that direct expression of said transgene is selected from the groups consisting of:
a. one or more transcription initiation sequences (such as a promoter),
b. one or more translation initiation sequences,
c. one or more mRNA stability sequences,
d. one or more polyadenylation sequences,
e. one or more secretory sequences,
f. one or more enhancer sequences,
g. one or more introns,
h. one or more TATA boxes,
i. one or more microRNA targeted sequences,
j. one or more polylinker sequences facilitating the insertion of a DNA fragment within a vector,
k. one or more splicing signal sequences,
l. one or more transcription termination sequences (such as polyadenylation sequences) or
m. any combinations of any one or more of the groups a. to l.

As used herein, the term “promoter” refers to a regulatory element that directs the transcription of a transgene to which it is operably linked. A promoter can regulate both rate and efficiency of transcription of an operably linked transgene. A promoter may also be operably linked to other regulatory elements which enhance (“enhancers”) or repress (“repressors”) promoter-dependent transcription of a transgene. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is generally located near the transcription start site of the transgene to which is operably linked, on the same strand and upstream of the DNA sequence (towards the 5′ region of the sense strand). As used herein, the term “operably linked” refers to a linkage of elements in a functional relationship. A transgene is “operably linked” when it is placed into a functional relationship with another nucleic acid sequence. For instance, a promoter or transcription regulatory sequence is operably linked to a transgene if it affects the transcription of the transgene. Operably linked means that the DNA sequences being linked are typically contiguous.

Preferably, the one or more regulatory sequences according to the invention comprise in the heterologous nucleic acid are specifically selected to drive the expression of the transgene in the central nervous system (CNS). In one preferred embodiment, the one or more regulatory sequences comprise a neuronal promoter. Accordingly, the heterologous nucleic acid construct may comprise any regulatory elements such as any of those listed above, either alone or in any combinations of two or more elements, any combinations of three or more elements, any combinations of four or more elements, any combinations of five or more elements and so forth. In another embodiment, the one or more regulatory sequences comprise or consist of a promoter and a transcription termination sequence. In a further embodiment, the one or more regulatory sequences comprise or consist of a promoter, an enhancer (such as one or more introns) and a polyadenylation site. Generally, the heterologous nucleic acid comprises regulatory sequences preceding (5′ non-coding sequences) and following (3′ non-coding sequences) the coding sequence that are required for expression of the transgene. Thus, in specific embodiments, said heterologous nucleic acid comprises at least (i) a transgene under the control of (ii) a promoter and (iii) a 3′ untranslated region that usually contains a polyadenylation sequence/site and/or transcription terminator.

In another embodiment, the neuronal promoter comprises SEQ ID NO: 8, or a sequence having at least 95%, at least 96%, at least 97%, at least 98% or at least 99% sequence identity thereto. In another embodiment, the neuronal promoter can be combined to untranslated sequences, either naturally occurring or engineered such as any of SEQ ID NO: 9, 11, 12, 13, 14, or 16. In non-limiting examples, sequences combining a neuronal promoter and untranslated sequences comprise or consist of SEQ ID NO: 10 or a sequence having at least 95%, at least 96%, at least 97%, at least 98% or at least 99% sequence identity thereto, or SEQ ID No. 15 or a sequence having at least 95%, at least 96%, at least 97%, at least 98% or at least 99% sequence identity thereto.

As used herein, the term “polyadenylation signal” or “polyadenylation site” or “poly(A) signal/site” refers to a specific recognition sequence within 3′ untranslated region (3′ UTR) of the gene, which is transcribed into precursor mRNA molecule and guides the termination of the gene transcription. Poly(A) signal acts as a signal for the endonucleolytic cleavage of the newly formed precursor mRNA at its 3′-end, and for the addition to this 3′-end of an RNA stretch consisting only of adenine bases (polyadenylation process; poly(A) tail). Poly(A) tail is important for the nuclear export, translation, and stability of mRNA. In the context of the invention, the polyadenylation signal is a recognition sequence that can direct polyadenylation of mammalian genes and/or viral genes, in mammalian cells. In a further embodiment, the polyadenylation sequence comprises or consists of SEQ ID NO: 17 or a sequence having at least 95%, at least 96%, at least 97%, at least 98% or at least 99% sequence identity thereto.

Non-limiting example of a heterologous nucleic acid that can be packaged in the rAAV particles is a sequence comprising SEQ ID No. 18

Non-limiting embodiments of neurodegenerative diseases according to the present invention comprise slow and fast progressive neurodegenerative diseases, such as neuronal ceroid lipofuscinosis (such as NCL type 11), mucopolysaccharidosis (MPS) such as MPS IIIC (O'Leary C. et al., 2016), progressive supranuclear palsy (PSP), corticobasal syndrome, Parkinson's disease, dementia with Lewy bodies, Alzheimer's disease or amyotrophic lateral sclerosis with or without dementia (ALS or ALS-D).

In the context of the invention as a whole, the neurodegenerative disease is preferably an adult-onset neurodegenerative disease. Examples of preferred adult-onset neurodegenerative diseases that can be treated are fronto-temporal dementia (FTD), NCL type 11 and/or PGR haploinsufficiency.

A suitable dosage of the rAAV particle according to the present invention may be determined by a skilled practitioner. The selected dose will depend upon a variety of pharmacokinetic factors including the time of administration, the rate of spreading of the rAAV particle, the rate of expression of the transgene, the frequency of administration, the optional presence of other drugs, compounds and/or materials used in combination with the particular rAAV particles, the age, sex, weight, condition, general health and prior medical history of the patient being treated.

A suitable total dose of rAAV particles comprising AAVTT capsids to be administered according to the present invention as a whole is at least 1×10⁶ vg of rAAV particles. Preferably, a suitable total dose of rAAV particles comprising AAVTT capsids to be administered according to the present invention as a whole is in the range of about 1×10⁶ to about 1×10²⁰, in the range of about 1×10⁸ to about 1×10¹⁸, in the range of about 1×10¹⁰ to about 1×10¹⁶, or in the range of about 1×10¹² to about 1×10¹⁴ vg of rAAV particles.

In the context of the invention as a whole, the total dose of rAAV particles can be administered to the primate either as one single dose or as multidose (e.g. two administrations or more, three administrations or more, four administrations or more, six administrations or more and the like). When multidose are administered, they can be administered simultaneously or sequentially. They can target the putamen in one hemisphere only or the putamen in each of the two hemispheres. They are preferably administered to the putamen in each of the two hemispheres to allow a homogeneous distribution of the particles and/or homogeneous expression of the transgene comprised in the particles. When particles are administered in the two hemispheres, each one of the hemispheres may receive one or more doses. When the putamen of each hemisphere receives more than one dose, the doses are typically administered at different but yet intra-putaminal administration sites (such as two administration sites within the putamen if two doses are administered, three administration sites if three doses are administered, four administration sites if four doses are administered) to improve even more the homogeneous distribution of the particles/protein to be expressed by the transgene incorporated in the particle).

For example, for a total dose of rAAV particles to be administered, two equal doses of rAAV particles can be administered each in the putamen of the right hemisphere and in the putamen of the left hemisphere, so that equal doses equivalent to half of the total dose is administered per each hemisphere. Alternative, the total dose can be split as desired across the putamen of each hemisphere.

In the context of the invention as a whole, the primate is a human or a non-human primate. Alternatively, the term patient or subject can be used interchangeably instead of primate.

The recombinant adeno-associated viral (rAAV) particles according to the present invention may be comprised in a pharmaceutical composition along with one or more carriers and/or excipients. The term carrier includes any and all solvents, dispersion media, isotonic and absorption delaying agents, and the like as long as they are physiologically compatible and are suitable for administration to the central nervous system of a primate in the context of the present invention. Examples of carriers include one or more of water, saline, phosphate buffered saline, buffers and the like, as well as combinations thereof.

The rAAV particle(s) according to the present invention may be produced by means of conventional methods and protocols. Briefly, viral particles can be produced in a host cell, more particularly in specific virus-producing cell (packaging cell), which is transfected with the appropriate heterologous nucleic acid to be packaged, in the presence of a helper vector or virus or other DNA construct(s). The term “packaging cells” as used herein, refers to a cell or cell line which may be transfected with a heterologous nucleic acid of the disclosure, through an appropriate plasmid, and provides in trans all the missing functions which are required for the complete replication and packaging of a viral particle.

Typically, a process of producing viral particles comprises the following steps: a) culturing a packaging cell comprising a nucleic acid construct or viral vector as described above in a culture medium; b) harvesting the viral particles from the cell culture supernatant and/or inside the cells; c) purifying the viral particles, typically via at least affinity chromatography and/or ion chromatography; and d) optionally formulating the viral particles to obtain a pharmaceutical composition.

The present invention also encompasses a method of delivering a therapeutic amount of a heterologous nucleic acid to the central nervous system (CNS) of a primate, wherein the heterologous nucleic acid is packaged in a recombinant adeno-associated virus (rAAV) particle comprising an AAV true type capsid comprises or consisting of SEQ ID NO: 1 and wherein the heterologous nucleic acid comprises: i) a 5′ inverted terminal repeat (ITR) or a fragment thereof and a 3′ ITR or a fragment thereof; ii) a transgene; and iii) one or more regulatory sequences that direct expression of said transgene; wherein the method comprises administering the rAAV particle to the putamen of said primate, and wherein the transgene encodes a secreted lysosomal protein, preferably human progranulin (PGRN), an active fragment thereof and/or an active variant thereof. The present invention also encompasses a method of expressing a transgene in the central nervous system (CNS) of a primate, wherein the transgene is comprised in an heterologous nucleic acid packaged in recombinant adeno-associated virus (rAAV) particles comprising an AAV true type capsid comprises or consisting of SEQ ID NO: 1, wherein the heterologous nucleic acid further comprises: i) a 5′ inverted terminal repeat (ITR) or a fragment thereof and a 3′ ITR or a fragment thereof; and ii) one or more regulatory sequences that direct expression of said transgene; wherein the method comprises administering the rAAV particles to the putamen of said primate and wherein further to the spreading of the rAAV particles within the CNS, the transgene packaged within the particles can be expressed, and wherein the transgene encodes a secreted lysosomal protein, preferably human progranulin (PGRN), an active fragment thereof and/or an active variant thereof. The present invention also encompasses a pharmaceutical composition comprising a recombinant adeno-associated viral (rAAV) particle and one or more carriers and/or excipients, wherein the rAAV particle comprises: a) an AAV true type capsid comprising or consisting of SEQ ID NO: 1 and b) a heterologous nucleic acid packaged therein; wherein the heterologous nucleic acid comprises: i. a 5′ inverted terminal repeat (ITR) or a fragment thereof and a 3′ ITR or a fragment thereof; ii. a transgene; and iii. one or more regulatory sequences that direct expression of said transgene; wherein the pharmaceutical composition is formulated for intra-putaminal administration to a primate in need thereof, and wherein the transgene encodes a secreted lysosomal protein, preferably human progranulin (PGRN), an active fragment thereof and/or an active variant thereof. Said composition is preferably for use in the treatment of a neurodegenerative disease in a primate.

### Description of the Sequences:

| SEQ | | |
|---|---|---|
| ID | | |
| NO: | Description | Sequence |
| 1 | AAVTT | MAADGYLPDWLEDTLSEGIRQWWKLKPGPPPPKPAERHKDDSRGLVLP |
| | | GYKYLGPFNGLDKGEPVNEADAAALEHDKAYDRQLDSGDNPYLKYNHA |
| | | DAEFQERLKEDTSFGGNLGRAVFQAKKRILEPLGLVEEPVKTAPGKKRPV |
| | | EHSPAEPDSSSGTGKSGQQPARKRLNFGQTGDADSVPDPQPLGQPPAA |
| | | PSGLGTNTMASGSGAPMADNNEGADGVGNSSGNWHCDSTWMGDRVIT |
| | | TSTRTWALPTYNNHLYKQISSQSGASNDNHYFGYSTPWGYFDFNRFHC |
| | | HFSPRDWQRLINNNWGFRPKRLSFKLFNIQVKEVTQNDGTTTIANNLTST |
| | | VQVFTDSEYQLPYVLGSAHQGCLPPFPADVFMVPQYGYLTLNNGSQAV |
| | | GRSSFYCLEYFPSQMLRTGNNFTFSYTFEDVPFHSSYAHSQSLDRLMNP |
| | | LIDQYLYYLSRTNTPSGTTTMSRLQFSQAGASDIRDQSRNWLPGPCYRQ |
| | | QRVSKTAADNNNSDYSWTGATKYHLNGRDSLVNPGPAMASHKDDEEKY |
| | | FPQSGVLIFGKQDSGKTNVDIEKVMITDEEEIRTTNPVATEQYGSVSTNLQ |
| | | SGNTQAATSDVNTQGVLPGMVWQDRDVYLQGPIWAKIPHTDGHFHPSP |
| | | LMGGFGLKHPPPQILIKNTPVPANPSTTFSAAKFASFITQYSTGQVSVEIE |
| | | WELQKENSKRWNPEIQYTSNYNKSVNVDFTVDTNGVYSEPRPIGTRYLT |
| | | RNL |
| 2 | 5′ ITR v1 | GCGCGCTCGCTCGCTCACTGAGGCCGCCCGGGCAAAGCCCGGGCGT |
| | | CGGGCGACCTTTGGTCGCCCGGCCTCAGTGAGCGAGCGAGCGCGCA |
| | | GAGAGGGAGTGGCCAACTCCATCACTAGGGGTTCCT |
| 3 | 3′ ITR v1 | AGGAACCCCTAGTGATGGAGTTGGCCACTCCCTCTCTGCGCGCTCGC |
| | | TCGCTCACTGAGGCCGGGCGACCAAAGGTCGCCCGACGCCCGGGCT |
| | | TTGCCCGGGCGGCCTCAGTGAGCGAGCGAGCGCGC |
| 4 | Transgene 1 | MWTLVSWVALTAGLVAGTRCPDGQFCPVACCLDPGGASYSCCRPLLDK |
| | | WPTTLSRHLGGPCQVDAHCSAGHSCIFTVSGTSSCCPFPEAVACGDGH |
| | | HCCPRGFHCSADGRSCFQRSGNNSVGAIQCPDSQFECPDFSTCCVMVD |
| | | GSWGCCPMPQASCCEDRVHCCPHGAFCDLVHTRCITPTGTHPLAKKLP |
| | | AQRTNRAVALSSSVMCPDARSRCPDGSTCCELPSGKYGCCPMPNATCC |
| | | SDHLHCCPQDTVCDLIQSKCLSKENATTDLLTKLPAHTVGDVKCDMEVS |
| | | CPDGYTCCRLQSGAWGCCPFTQAVCCEDHIHCCPAGFTCDTQKGTCEQ |
| | | GPHQVPWMEKAPAHLSLPDPQALKRDVPCDNVSSCPSSDTCCQLTSGE |
| | | WGCCPIPEAVCCSDHQHCCPQGYTCVAEGQCQRGSEIVAGLEKMPARR |
| | | ASLSHPRDIGCDQHTSCPVGQTCCPSLGGSWACCQLPHAVCCEDRQHC |
| | | CPAGYTCNVKARSCEKEVVSAQPATFLARSPHVGVKDVECGEGHFCHD |
| | | NQTCCRDNRQGWACCPYRQGVCCADRRHCCPAGFRCAARGTKCLRR |
| | | EAPRWDAPLRDPALRQLL |
| 5 | 5′ ITR v2 | TTGGCCACTCCCTCTCTGCGCGCTCGCTCGCTCACTGAGGCCGGG |
| | | CGACCAAAGGTCGCCCGACGCCCGGGCTTTGCCCGGGCGGCCTC |
| | | AGTGAGCGAGCGAGCGCGCAGAGAGGGAGTGGCCAACTCCATC |
| | | ACTAGGGGTTCCT |
| 6 | 3′ ITR v2 | AGGAACCCCTAGTGATGGAGTTGGCCACTCCCTCTCTGCGCGCTCGC |
| | | TCGCTCACTGAGGCCGGGCGACCAAAGGTCGCCCGACGCCCGGGCT |
| | | TTGCCCGGGCGGCCTCAGTGAGCGAGCGAGCGCGCAGAGAGGGAG |
| | | TGGCCAA |
| 7 | Transgene 1 | ATGTGGACCCTGGTGAGCTGGGTGGCCTTAACAGCAGGGCTGGTGG |
| | | CTGGAACGCGGTGCCCAGATGGTCAGTTCTGCCCTGTGGCCTGCTGC |
| | | CTGGACCCCGGAGGAGCCAGCTACAGCTGCTGCCGTCCCCTTCTGG |
| | | ACAAATGGCCCACAACACTGAGCAGGCATCTGGGTGGCCCCTGCCAG |
| | | GTTGATGCCCACTGCTCTGCCGGCCACTCCTGCATCTTTACCGTCTCA |
| | | GGGACTTCCAGTTGCTGCCCCTTCCCAGAGGCCGTGGCATGCGGGG |
| | | ATGGCCATCACTGCTGCCCACGGGGCTTCCACTGCAGTGCAGACGG |
| | | GCGATCCTGCTTCCAAAGATCAGGTAACAACTCCGTGGGTGCCATCC |
| | | AGTGCCCTGATAGTCAGTTCGAATGCCCGGACTTCTCCACGTGCTGT |
| | | GTTATGGTCGATGGCTCCTGGGGGTGCTGCCCCATGCCCCAGGCTTC |
| | | CTGCTGTGAAGACAGGGTGCACTGCTGTCCGCACGGTGCCTTCTGCG |
| | | ACCTGGTTCACACCCGCTGCATCACACCCACGGGCACCCACCCCCTG |
| | | GCAAAGAAGCTCCCTGCCCAGAGGACTAACAGGGCAGTGGCCTTGTC |
| | | CAGCTCGGTCATGTGTCCGGACGCACGGTCCCGGTGCCCTGATGGTT |
| | | CTACCTGCTGTGAGCTGCCCAGTGGGAAGTATGGCTGCTGCCCAATG |
| | | CCCAACGCCACCTGCTGCTCCGATCACCTGCACTGCTGCCCCCAAGA |
| | | CACTGTGTGTGACCTGATCCAGAGTAAGTGCCTCTCCAAGGAGAACG |
| | | CTACCACGGACCTCCTCACTAAGCTGCCTGCGCACACAGTGGGGGAT |
| | | GTGAAATGTGACATGGAGGTGAGCTGCCCAGATGGCTATACCTGCTG |
| | | CCGTCTACAGTCGGGGGCCTGGGGCTGCTGCCCTTTTACCCAGGCTG |
| | | TGTGCTGTGAGGACCACATACACTGCTGTCCCGCGGGGTTTACGTGT |
| | | GACACGCAGAAGGGTACCTGTGAACAGGGGCCCCACCAGGTGCCCT |
| | | GGATGGAGAAGGCCCCAGCTCACCTCAGCCTGCCAGACCCACAAGC |
| | | CTTGAAGAGAGATGTCCCCTGTGATAATGTCAGCAGCTGTCCCTCCTC |
| | | CGATACCTGCTGCCAACTCACGTCTGGGGAGTGGGGCTGCTGTCCAA |
| | | TCCCAGAGGCTGTCTGCTGCTCGGACCACCAGCACTGCTGCCCCCAG |
| | | GGCTACACGTGTGTAGCTGAGGGGCAGTGTCAGCGAGGAAGCGAGA |
| | | TCGTGGCTGGACTGGAGAAGATGCCTGCCCGCCGGGCTTCCTTATCC |
| | | CACCCCAGAGACATCGGCTGTGACCAGCACACCAGCTGCCCGGTGG |
| | | GGCAGACCTGCTGCCCGAGCCTGGGTGGGAGCTGGGCCTGCTGCCA |
| | | GTTGCCCCATGCTGTGTGCTGCGAGGATCGCCAGCACTGCTGCCCG |
| | | GCTGGCTACACCTGCAACGTGAAGGCTCGATCCTGCGAGAAGGAAGT |
| | | GGTCTCTGCCCAGCCTGCCACCTTCCTGGCCCGTAGCCCTCACGTGG |
| | | GTGTGAAGGACGTGGAGTGTGGGGAAGGACACTTCTGCCATGATAAC |
| | | CAGACCTGCTGCCGAGACAACCGACAGGGCTGGGCCTGCTGTCCCT |
| | | ACCGCCAGGGCGTCTGTTGTGCTGATCGGCGCCACTGCTGTCCTGCT |
| | | GGCTTCCGCTGCGCAGCCAGGGGTACCAAGTGTTTGCGCAGGGAGG |
| | | CCCCGCGCTGGGACGCCCCTTTGAGGGACCCAGCCTTGAGACAGCT |
| | | GCTGTGA |
| 8 | Promoter 1 | AGCTGAATGGGGTCCGCCTCTTTTCCCTGCCTAAACAGACAGGAACT |
| | | CCTGCCAATTGAGGGCGTCACCGCTAAGGCTCCGCCCCAGCCTGGG |
| | | CTCCACAACCAATGAAGGGTAATCTCGACAAAGAGCAAGGGGTGGGG |
| | | CGCGGGCGCGCAGGTGCAGCAGCACACAGGCTGGTCGGGAGGGCG |
| | | GGGCGCGACGTCTGCCGTGCGGGGTCCCGGCATCGGTTGCGCGC |
| 9 | Untranslated | GCGCTCCCTCCTCTCGGAGAGAGGGCTGTGGTAAAACCCGTCCGGAA |
| | 5′ sequence | ATTGGCCGCCGCTGCCGCCACCGCCGCCGCCGCCGCCGCGCCGAG |
| | | CGGAGGAGGAGGAGGAGGCGAGGAGGAGAGACTGTGAGTGGGACC |
| | | GCCAAGGCCGCGGGGGGGGACCCTTGCTGGGGGGCGGGTAGGGGC |
| | | GGGACGTGGCGCGGGAGGGGCCCGCGGGGTCGGGCGACACGGCT |
| | | GGCGGTTGGCGTCCCTCCTCTCTACCCTCCCCCTCCCTCTGCCGCCG |
| | | GTGGTGGCTTTCTCCACTCGTCTCCCGCAATCGCGAGCGACGGTTCT |
| | | CAGCGCGATCTCCCTGGAGCCACCTTCGATTGACGCCCTCCCGCTGC |
| | | CCGCCCCATCTGTGCGCATCCTAGGCCCCAGCTGTGCAAGCGCCCTT |
| | | GTCGTCTGGGCTTCGCCAGTTGGGGCTGCGCGCGCTCCTGCCCTTCT |
| | | TGGGGCTTTGGGCCTCGGCACTGTCGCGCGCCCGCGGTCCCGGCCT |
| | | CTCCCTGGATCGCGCTGTCCCCTTCTCCCTCGCGCGCCCCCACTCCC |
| | | GTTACTTGCTCCCCCCTCACACACACAGACTGGCGCGCGTGCGCAGT |
| | | CCATCTCCCGTTGGGAGAGTGCGCCACAAGGGCTCCTGAGCTCTTAC |
| | | CCCCATCTCTGGGTTTTGCTCCCTCCTCCTCCTCTCCCATTCCGTGAC |
| | | TTTTTGCCCCCACTGCAAGCGAGTCGGTCCATCAGCTCCATTCCCCAC |
| | | TTGGCAGGAACAAGTTGAGGGTTATTGTCCACCCACAAAAAGGACTA |
| | | GACATTTTGTTCCTAGGTCCCACAACTCATCATAAAGAGTTGGTTGTA |
| | | GTTCTCATCAGGAACCGTGGGCAAGGGACTGTGCGTTCCTCAGCACT |
| | | CGAAGCTCTTCCGTGAGACCTTGCCCGCAGGGTGCTCTGGTTCTTTG |
| | | GGGTTGCTGTGCTGTGGCTTCGGAATTTGAGCGTCTTCCCACCCTCC |
| | | CTCCCCTCCCTTCGCCAGCGTTCTGTCTACAAGAAAGAATAGGCAGGT |
| | | GTCCTTGGATATCGTAGTTGCTAATCGCCTATACACTGTTCTATTACAC |
| | | CTTTCTGCTAAGGATAGGGTTTTTGGTTTTGGTTTTGGTTTTGTTCCCC |
| | | ACCCTCCAGTTTGGTTTAGTTTTGGTTTTGGCATTTAGGGTTTTTTGGG |
| | | GGGGAGTAATATCTTGTGGTAAAGACCCATCTGACCCAAGATACCTTT |
| | | TTTCTCATACTGGAACCCTAGGCAGCAGTTGCTATTTCCCTGAGTTAG |
| | | CAATAGTTTTACAGTATTTTGAGGCCTTTTGTCCATAATTCTCACGGAA |
| | | TCCCTCAGGGATCAGATTAGCTGCTGTTGGGATCAGGAAATTGGGTTA |
| | | CACCGCTGAAATCTCTTGCTGGGGCCCTTGTTTTGAATTGGAAAGTCA |
| | | GGAGGCTGGAACGAAGGCTCACAAGTTAACAGTGCCAGCTGCTCTTC |
| | | CAGAAGCCCTGGATTCAGTCCCACCAATCCATCGCGGGTCACAACCA |
| | | TCTGTAACTTCAGTCCCAAGGGGTCCGAAGCCCTCTTCTGGCTTTGCC |
| | | CTATTATTTTATTTATCTTATCTGTTTTTGTCTTGTCATCTGGCAAGCCC |
| | | AGGGGGCCATTGGGTGCAACTTATAAACTGACTTCTGTATCTTAAGAA |
| | | GCCAACCATACAGTGCTTACATTCCAGAAAAAAAATCTGCCACTTTAA |
| | | CAGCACTAGAACTAGGGTTTAGAGAAGTATCATAAAGGTCAAATATCT |
| | | TTGACCAATATCACCAGCAACCTAAAGCTGTTAAGAAATCTTTGGGCC |
| | | CCAGCTTGACCCAAGGATACAGTATCCTAGGGAAGTTACCAAAATCAG |
| | | AGATAGTATGCAGCAGCCAGGGGTCTCATGTGTGGCACTCAAGCTCA |
| | | CCTATACTCACTACTGTGCAGACAGCTGTGTTCTCTGTAATACTTACAT |
| | | ATTTGTTTAATACTTCAGGGAGGAAAAGTCAGAAGACCAGGATCTCCA |
| | | GGGCCTCA |
| 10 | Promoter 2 | AGCTGAATGGGGTCCGCCTCTTTTCCCTGCCTAAACAGACAGGAACT |
| | | CCTGCCAATTGAGGGCGTCACCGCTAAGGCTCCGCCCCAGCCTGGG |
| | | CTCCACAACCAATGAAGGGTAATCTCGACAAAGAGCAAGGGGTGGGG |
| | | CGCGGGCGCGCAGGTGCAGCAGCACACAGGCTGGTCGGGAGGGCG |
| | | GGGCGCGACGTCTGCCGTGCGGGGTCCCGGCATCGGTTGCGCGCAC |
| | | CGGTGCGCTCCCTCCTCTCGGAGAGAGGGCTGTGGTAAAACCCGTCC |
| | | GGAAATTGGCCGCCGCTGCCGCCACCGCCGCCGCCGCCGCCGCGC |
| | | CGAGCGGAGGAGGAGGAGGAGGCGAGGAGGAGAGACTGTGAGTGG |
| | | GACCGCCAAGGCCGCGGGGGGGGACCCTTGCTGGGGGGGGGTAG |
| | | GGGCGGGACGTGGCGCGGGAGGGGCCCGCGGGGTCGGGCGACAC |
| | | GGCTGGCGGTTGGCGTCCCTCCTCTCTACCCTCCCCCTCCCTCTGCC |
| | | GCCGGTGGTGGCTTTCTCCACTCGTCTCCCGCAATCGCGAGCGACGG |
| | | TTCTCAGCGCGATCTCCCTGGAGCCACCTTCGATTGACGCCCTCCCG |
| | | CTGCCCGCCCCATCTGTGCGCATCCTAGGCCCCAGCTGTGCAAGCGC |
| | | CCTTGTCGTCTGGGCTTCGCCAGTTGGGGCTGCGCGCGCTCCTGCC |
| | | CTTCTTGGGGCTTTGGGCCTCGGCACTGTCGCGCGCCCGCGGTCCC |
| | | GGCCTCTCCCTGGATCGCGCTGTCCCCTTCTCCCTCGCGCGCCCCCA |
| | | CTCCCGTTACTTGCTCCCCCCTCACACACACAGACTGGCGCGCGTGC |
| | | GCAGTCCATCTCCCGTTGGGAGAGTGCGCCACAAGGGCTCCTGAGCT |
| | | CTTACCCCCATCTCTGGGTTTTGCTCCCTCCTCCTCCTCTCCCATTCC |
| | | GTGACTTTTTGCCCCCACTGCAAGCGAGTCGGTCCATCAGCTCCATTC |
| | | CCCACTTGGCAGGAACAAGTTGAGGGTTATTGTCCACCCACAAAAAG |
| | | GACTAGACATTTTGTTCCTAGGTCCCACAACTCATCATAAAGAGTTGG |
| | | TTGTAGTTCTCATCAGGAACCGTGGGCAAGGGACTGTGCGTTCCTCA |
| | | GCACTCGAAGCTCTTCCGTGAGACCTTGCCCGCAGGGTGCTCTGGTT |
| | | CTTTGGGGTTGCTGTGCTGTGGCTTCGGAATTTGAGCGTCTTCCCACC |
| | | CTCCCTCCCCTCCCTTCGCCAGCGTTCTGTCTACAAGAAAGAATAGGC |
| | | AGGTGTCCTTGGATATCGTAGTTGCTAATCGCCTATACACTGTTCTATT |
| | | ACACCTTTCTGCTAAGGATAGGGTTTTTGGTTTTGGTTTTGGTTTTGTT |
| | | CCCCACCCTCCAGTTTGGTTTAGTTTTGGTTTTGGCATTTAGGGTTTTT |
| | | TGGGGGGGAGTAATATCTTGTGGTAAAGACCCATCTGACCCAAGATA |
| | | CCTTTTTTCTCATACTGGAACCCTAGGCAGCAGTTGCTATTTCCCTGA |
| | | GTTAGCAATAGTTTTACAGTATTTTGAGGCCTTTTGTCCATAATTCTCA |
| | | CGGAATCCCTCAGGGATCAGATTAGCTGCTGTTGGGATCAGGAAATT |
| | | GGGTTACACCGCTGAAATCTCTTGCTGGGGCCCTTGTTTTGAATTGGA |
| | | AAGTCAGGAGGCTGGAACGAAGGCTCACAAGTTAACAGTGCCAGCTG |
| | | CTCTTCCAGAAGCCCTGGATTCAGTCCCACCAATCCATCGCGGGTCA |
| | | CAACCATCTGTAACTTCAGTCCCAAGGGGTCCGAAGCCCTCTTCTGG |
| | | CTTTGCCCTATTATTTTATTTATCTTATCTGTTTTTGTCTTGTCATCTGG |
| | | CAAGCCCAGGGGGCCATTGGGTGCAACTTATAAACTGACTTCTGTATC |
| | | TTAAGAAGCCAACCATACAGTGCTTACATTCCAGAAAAAAAATCTGCC |
| | | ACTTTAACAGCACTAGAACTAGGGTTTAGAGAAGTATCATAAAGGTCA |
| | | AATATCTTTGACCAATATCACCAGCAACCTAAAGCTGTTAAGAAATCTT |
| | | TGGGCCCCAGCTTGACCCAAGGATACAGTATCCTAGGGAAGTTACCA |
| | | AAATCAGAGATAGTATGCAGCAGCCAGGGGTCTCATGTGTGGCACTC |
| | | AAGCTCACCTATACTCACTACTGTGCAGACAGCTGTGTTCTCTGTAAT |
| | | ACTTACATATTTGTTTAATACTTCAGGGAGGAAAAGTCAGAAGACCAG |
| | | GATCTCCAGGGCCTCA |
| 11 | Untranslated | GCGCTCCCTCCTCTCGGAGAGAGGGCTGTGGTAAAACCCGTCCGGAA |
| | 5′ sequence | ATTGGCCGCCGCTGCCGCCACCGCCGCCGCCGCCGCCGCGCCGAG |
| | | CGGAGGAGGAGGAGGAGGCGAGGAGGAGAGACT |
| 12 | Untranslated | GTGAGTGGGACCGCCAAGGCCGCGGGGGGGGACCCTTGCTGGGGG |
| | 5′ sequence | GCGGGTAGGGGGGGGACGTGGCGCGGGAGGGGCCCGCGGGGTCG |
| | | GGCGACACGGCTGGCGGTTGGCGTCCCTCCTCTCTACCCTCCCCCTC |
| | | CCTCTGCCGCCGGTGGTGGCTTTCTCCACTCGTCTCCCGCAATCGCG |
| | | AGCGACGGTTCTCAGCGCGATCTCCCTGGAGCCACCTTCGATTGACG |
| | | CCCTCCCGCTGCCCGCCCCATCTGTGCGCATCCTAGGCCCCAGCTGT |
| | | GCAAGCGCCCTTGTCGTCTGGGCTTCGCCAGTTGGGGCTGCGCGCG |
| | | CTCCTGCCCTTCTTGGGGCTTTGGGCCTCGGCACTGTCGCGCGCCCG |
| | | CGGTCCCGGCCTCTCCCTGGATCGCGCTGTCCCCTTCTCCCTCGCGC |
| | | GCCCCCACTCCCGTTACTTGCTCCCCCCTCACACACACAGACTGGCG |
| | | CGCGTGCGCAGTCCATCTCCCGTTGGGAGAGTGCGCCACAAGGGCT |
| | | CCTGAGCTCTTACCCCCATCTCTGGGTTTTGCTCCCTCCTCCTCCTCT |
| | | CCCATTCCGTGACTTTTTGCCCCCACTGCAAGCGAGTCGGTCCATCA |
| | | GCTCCATTCCCCACTTGGCAGGAACAAGTTGAGGGTTATTGTCCACCC |
| | | ACAAAAAGGACTAGACATTTTGTTCCTAGGTCCCACAACTCATCATAAA |
| | | GAGTTGGTTGTAGTTCTCATCAGGAACCGTGGGCAAGGGACTGTGCG |
| | | TTCCTCAGCACTCGAAGCTCTTCCGTGAGACCTTGCCCGCAGGGTGC |
| | | TCTGGTTCTTTGGGGTTGCTGTGCTGTGGCTTCGGAATTTGAGCGTCT |
| | | TCCCACCCTCCCTCCCCTCCCTTCGCCAGCG |
| 13 | Untranslated | TTCTGTCTACAAGAAAGAATAGGCAGGTGTCCTTGGATATCGTAGTTG |
| | 3′ sequence | CTAATCGCCTATACACTGTTCTATTACACCTTTCTGCTAAGGATAGGGT |
| | | TTTTGGTTTTGGTTTTGGTTTTGTTCCCCACCCTCCAGTTTGGTTTAGT |
| | | TTTGGTTTTGGCATTTAGGGTTTTTTGGGGGGGAGTAATATCTTGTGG |
| | | TAAAGACCCATCTGACCCAAGATACCTTTTTTCTCATACTGGAACCCTA |
| | | GGCAGCAGTTGCTATTTCCCTGAGTTAGCAATAGTTTTACAGTATTTTG |
| | | AGGCCTTTTGTCCATAATTCTCACGGAATCCCTCAGGGATCAGATTAG |
| | | CTGCTGTTGGGATCAGGAAATTGGGTTACACCGCTGAAATCTCTTGCT |
| | | GGGGCCCTTGTTTTGAATTGGAAAGTCAGGAGGCTGGAACGAAGGCT |
| | | CACAAGTTAACAGTGCCAGCTGCTCTTCCAGAAGCCCTGGATTCAGTC |
| | | CCACCAATCCATCGCGGGTCACAACCATCTGTAACTTCAGTCCCAAG |
| | | GGGTCCGAAGCCCTCTTCTGGCTTTGCCCTATTATTTTATTTATCTTAT |
| | | CTGTTTTTGTCTTGTCATCTGGCAAGCCCAGGGGGCCATTGGGTGCA |
| | | ACTTATAAACTGACTTCTGTATCTTAAGAAGCCAACCATACAGTGCTTA |
| | | CATTCCAGAAAAAAAATCTGCCACTTTAACAGCACTAGAACTAGGGTT |
| | | TAGAGAAGTATCATAAAGGTCAAATATCTTTGACCAATATCACCAGCAA |
| | | CCTAAAGCTGTTAAGAAATCTTTGGGCCCCAGCTTGACCCAAGGATAC |
| | | AGTATCCTAGGGAAGTTACCAAAATCAGAGATAGTATGCAGCAGCCAG |
| | | GGGTCTCATGTGTGGCACTCAAGCTCACCTATACTCACTACTGTGCAG |
| | | ACAGCTGTGTTCTCTGTAATACTTACATATTTGTTTAATACTTCAG |
| 14 | Untranslated | GGAGGAAAAGTCAGAAGACCAGGATCTCCAGGGCCTCA |
| | 3′ sequence | |
| 15 | Promoter 3 | CTCTACCATTACGTTTTATCCTCAGACTCTATCTCCCCATTTTAAAGGA |
| | | ATATTATTTTTAAATGCTACACTCTCATTTTTTAAATGGCTCCTTTTAATT |
| | | CTACTGCTAAAATACTTTTGGTACAATATGCCTTTTTTTCTATTTTTTTTT |
| | | TTTAGTGCAAGTATAAAATATGTCATTTAAATCTCTTTTATCTAATTTAA |
| | | GGAACTTAAGATTTTCTTCCCAAAATTTCACAAGGTAGGAAAATGATGT |
| | | ACTTTATTTTTGTGAATGATATTGCACTGTAGATCTTGCTGTTTCTTGCT |
| | | TTGTTCTCAATTAAATATCATGTTTCCTCTACAGCTTTATAGACATTTTG |
| | | ATCAGATTAAGGACATTCTAATTATAGTTCTTTAAGGTGTTTTTAAAATC |
| | | ATATGTAGGTATTGAATTTTATTAAATGCTTTTTCCTGCATGTATTGGGA |
| | | TACTCACATGATCTGTTCTTAAAATTATAATTGATTTCTAATTTTAAACC |
| | | ATCCTTGCATTCGTGGAATAAAACTCAGCCTGAAGGTGTGGCTGGAG |
| | | AGATGGCTTGTTGCTCTTGCAGAGGACCCAAGTTCAGATCCTATTCTC |
| | | TGTATATGCAAATACCTGTATTCTCACACCCCAACATACACACACACAC |
| | | ACACACACACACACACACACACTACCACTCTTACCCACTTGTTTTTTAC |
| | | ATTTCTATCTTAGTATTTTATGTGTATAAGTGTTTTGCCTGGTGCTCACT |
| | | GTGGTCAGAAGAGGGCACAGGATTCCCTGAGACTAGAATTACAAATG |
| | | GTTTAGAATGGCCATATGGGAAATCCTCTAGATTTCCCCCACTGTAGT |
| | | AAGATATTCACTTAGGTGATCCTGTCCCAAAGTCAGCCATCATCCTTAT |
| | | TTTTTTTTTTCTTTCCATCTGATATCCAATACTTTTGGCTCAATTTTTAA |
| | | CATAAATCTTAACTATCACAACTTATCAGATTTCAACTGCTACTGTCCT |
| | | GGTTAAAGCCTTCATCATCTATCTTTCTTCAACTGCTGCCAGGACCTCT |
| | | GGACCAGCCAGTTCTTCATTCTTCACTGGCAACATAGGTTTTATGGTG |
| | | ACAGCTAGTGACTCAAATATTTATCAAGGGCTTCTCATCTCAAAATAAT |
| | | CTCCTAGTTCTTTTGGTGGCCTAGGTCTCTCTCCAGTCACACTGGCCT |
| | | CCTTAGTAAGGCAGGCATAGTCCTTCCTTAGAGTGTTTAAACTTGCCT |
| | | AGAATGTTTTCCCCAATTACCCATATTGGGAGACGACATGAGGGCAAA |
| | | AGCTAGAGGGTATCATAATAGCACTTCTTTTGTCCTTGCCCTATCTATT |
| | | TCAAAGTCTTTATCTCTGTGCAAAATTTTAAGTTCTACTTTCTTGTATGT |
| | | TTAGTATGACTCTTCCTTACCAGGAGTCTAGTTTGTCTCCTTGTTCAGT |
| | | ACTAAAACAGTGCCTAGCAAATAAATGAATAGAGAGGGGAGCCAAATT |
| | | TGAATCAGAAAGTCTCTTGTTGCATAGTGTTTAAAAAACAAACAAAGAA |
| | | AGAAAGTCTCTTGTTGAGCATTTGTTTAGCACAAAGAGCATTGGATGC |
| | | TGACTGGTATCAGGGTAAGGCTGCTTTGACAATGCTCCCTCTGGCCTC |
| | | ACTCCCTTTTATACGTACTTCCATCAAACCATCTGATTCAACAATGACA |
| | | GACCGATCTCTTATGGGCTTGGCACACACCATCTGCCCATTATAAACG |
| | | TCTGCAAAGACCAAGGTTTGATATGTTGATTTTACTGTCAGCCTTAAGA |
| | | GTGCGACATCTGCTAATTTAGTGTAATAATACAATCAGTAGACCCTTTA |
| | | AAACAAGTCCCTTGGCTTGGAACAACGCCAGGCTCCTCAACAGGCAA |
| | | CTTTGCTACTTCTACAGAAAATGATAATAAAGAAATGCTGGTGAAGTCA |
| | | AATGCTTATCACAATGGTGAACTACTCAGCAGGGAGGCTCTAATAGGC |
| | | GCCAAGAGCCTAGACTTCCTTAAGCGCCAGAGTCCACAAGGGCCCAG |
| | | TTAATCCTCAACATTCAAATGCTGCCCACAAAACCAGCCCCTCTGTGC |
| | | CCTAGCCGCCTCTTTTTTCCAAGTGACAGTAGAACTCCACCAATCCGC |
| | | TTAATTAAAGCTGAATGGGGTCCGCCTCTTTTCCCTGCCTAAACAGAC |
| | | AGGAACTCCTGCCAATTGAGGGCGTCACCGCTAAGGCTCCGCCCCAG |
| | | CCTGGGCTCCACAACCAATGAAGGGTAATCTCGACAAAGAGCAAGGG |
| | | GTGGGGCGCGGGCGCGCAGGTGCAGCAGCACACAGGCTGGTCGGG |
| | | AGGGGGGGGCGCGACGTCTGCCGTGCGGGGTCCCGGCATCGGTTG |
| | | CGCGC |
| 16 | Untranslated | CTCTACCATTACGTTTTATCCTCAGACTCTATCTCCCCATTTTAAAGGA |
| | 3′ sequence | ATATTATTTTTAAATGCTACACTCTCATTTTTTAAATGGCTCCTTTTAATT |
| | | CTACTGCTAAAATACTTTTGGTACAATATGCCTTTTTTTCTATTTTTTTTT |
| | | TTTAGTGCAAGTATAAAATATGTCATTTAAATCTCTTTTATCTAATTTAA |
| | | GGAACTTAAGATTTTCTTCCCAAAATTTCACAAGGTAGGAAAATGATGT |
| | | ACTTTATTTTTGTGAATGATATTGCACTGTAGATCTTGCTGTTTCTTGCT |
| | | TTGTTCTCAATTAAATATCATGTTTCCTCTACAGCTTTATAGACATTTTG |
| | | ATCAGATTAAGGACATTCTAATTATAGTTCTTTAAGGTGTTTTTAAAATC |
| | | ATATGTAGGTATTGAATTTTATTAAATGCTTTTTCCTGCATGTATTGGGA |
| | | TACTCACATGATCTGTTCTTAAAATTATAATTGATTTCTAATTTTAAACC |
| | | ATCCTTGCATTCGTGGAATAAAACTCAGCCTGAAGGTGTGGCTGGAG |
| | | AGATGGCTTGTTGCTCTTGCAGAGGACCCAAGTTCAGATCCTATTCTC |
| | | TGTATATGCAAATACCTGTATTCTCACACCCCAACATACACACACACAC |
| | | ACACACACACACACACACACACTACCACTCTTACCCACTTGTTTTTTAC |
| | | ATTTCTATCTTAGTATTTTATGTGTATAAGTGTTTTGCCTGGTGCTCACT |
| | | GTGGTCAGAAGAGGGCACAGGATTCCCTGAGACTAGAATTACAAATG |
| | | GTTTAGAATGGCCATATGGGAAATCCTCTAGATTTCCCCCACTGTAGT |
| | | AAGATATTCACTTAGGTGATCCTGTCCCAAAGTCAGCCATCATCCTTAT |
| | | TTTTTTTCTTTCTTTCCATCTGATATCCAATACTTTTGGCTCAATTTTTAA |
| | | CATAAATCTTAACTATCACAACTTATCAGATTTCAACTGCTACTGTCCT |
| | | GGTTAAAGCCTTCATCATCTATCTTTCTTCAACTGCTGCCAGGACCTCT |
| | | GGACCAGCCAGTTCTTCATTCTTCACTGGCAACATAGGTTTTATGGTG |
| | | ACAGCTAGTGACTCAAATATTTATCAAGGGCTTCTCATCTCAAAATAAT |
| | | CTCCTAGTTCTTTTGGTGGCCTAGGTCTCTCTCCAGTCACACTGGCCT |
| | | CCTTAGTAAGGCAGGCATAGTCCTTCCTTAGAGTGTTTAAACTTGCCT |
| | | AGAATGTTTTCCCCAATTACCCATATTGGGAGACGACATGAGGGCAAA |
| | | AGCTAGAGGGTATCATAATAGCACTTCTTTTGTCCTTGCCCTATCTATT |
| | | TCAAAGTCTTTATCTCTGTGCAAAATTTTAAGTTCTACTTTCTTGTATGT |
| | | TTAGTATGACTCTTCCTTACCAGGAGTCTAGTTTGTCTCCTTGTTCAGT |
| | | ACTAAAACAGTGCCTAGCAAATAAATGAATAGAGAGGGGAGCCAAATT |
| | | TGAATCAGAAAGTCTCTTGTTGCATAGTGTTTAAAAAACAAACAAAGAA |
| | | AGAAAGTCTCTTGTTGAGCATTTGTTTAGCACAAAGAGCATTGGATGC |
| | | TGACTGGTATCAGGGTAAGGCTGCTTTGACAATGCTCCCTCTGGCCTC |
| | | ACTCCCTTTTATACGTACTTCCATCAAACCATCTGATTCAACAATGACA |
| | | GACCGATCTCTTATGGGCTTGGCACACACCATCTGCCCATTATAAACG |
| | | TCTGCAAAGACCAAGGTTTGATATGTTGATTTTACTGTCAGCCTTAAGA |
| | | GTGCGACATCTGCTAATTTAGTGTAATAATACAATCAGTAGACCCTTTA |
| | | AAACAAGTCCCTTGGCTTGGAACAACGCCAGGCTCCTCAACAGGCAA |
| | | CTTTGCTACTTCTACAGAAAATGATAATAAAGAAATGCTGGTGAAGTCA |
| | | AATGCTTATCACAATGGTGAACTACTCAGCAGGGAGGCTCTAATAGGC |
| | | GCCAAGAGCCTAGACTTCCTTAAGCGCCAGAGTCCACAAGGGCCCAG |
| | | TTAATCCTCAACATTCAAATGCTGCCCACAAAACCAGCCCCTCTGTGC |
| | | CCTAGCCGCCTCTTTTTTCCAAGTGACAGTAGAACTCCACCAATCCGC |
| | | TTAATTAA |
| 17 | Poly A | GATCCAGACATGATAAGATACATTGATGAGTTTGGACAAACCACAACT |
| | | AGAATGCAGTGAAAAAAATGCTTTATTTGTGAAATTTGTGATGCTATTG |
| | | CTTTATTTGTAACCATTATAAGCTGCAATAAACAAGTTAACAACAACAA |
| | | TTGCATTCATTTTATGTTTCAGGTTCAGGGGGAGGTGTGGGAGGTTTT |
| | | TTAG |
| 18 | Exemplary | GCGCGCTCGCTCGCTCACTGAGGCCGCCCGGGCAAAGCCCGGGCGTCGGGCGACC |
| | heterologous | TTTGGTCGCCCGGCCTCAGTGAGCGAGCGAGCGCGCAGAGAGGGAGTGGCCAACT |
| | nucleic acid | CCATCACTAGGGGTTCCTTGTAGTTAATGATTAACCTCTGctagcAGCTGAATGG |
| | | GGTCCGCCTCTTTTCCCTGCCTAAACAGACAGGAACTCCTGCCAATTGAGGGCGT |
| | | CACCGCTAAGGCTCCGCCCCAGCCTGGGCTCCACAACCAATGAAGGGTAATCTCG |
| | | ACAAAGAGCAAGGGGTGGGGCGCGGGCGCGCAGGTGCAGCAGCACACAGGCTGGT |
| | | CGGGAGGGCGGGGCGCGACGTCTGCCGTGCGGGGTCCCGGCATCGGTTGCGCGCA |
| | | CCGGTgcgctccctcctctcggagagagggctgtggtaaaacccgtccggaaaTt |
| | | ggccgccgctgccgccaccgccgccgccgccgccgcgccgagcggaggaggagga |
| | | ggaggcgaggaggagagactgtgagtgggaccgccaaggccgcgggcggggaccc |
| | | ttgctggggggcgggtaggggcgggacgtggcgcgggaggggcccgcggggtcgg |
| | | gcgacacggctggcggTtggcgtccctcctctctaccctccccctccctctgccg |
| | | ccggtggtggctttctccactcgtctcccgcaatcgcgagcgacggttctcagcg |
| | | cgatctccctggagccaccttcgaTtgacgccctcccgctgcccgccccatctgt |
| | | gcgcatcctaggccccagctgtgcaagcgcccttgtcgtctgggcttcgccagtt |
| | | ggggctgcgcgcgctcctgcccttcttggggctttgggcctcggcactgtcgcgc |
| | | gcccgcggtcccggcctctccctggatcgcgctgtccccttctccctcgcgcgcc |
| | | cccactcccgttacttgctcccccctcacacacacagactggcgcgcgtgcgcag |
| | | tccatctcccgttgggagagtgcgccacaagggctcctgagctcttacccccatc |
| | | tctgggttttgctccctcctcctcctctcccattccgtgactttttgcccccact |
| | | gcaagcgagtcggtccatcagctccattccccacttggcaggaacaagttgaggg |
| | | ttattgtccacccacaaaaaggactagacattTtgttcctaggtcccacaactca |
| | | tcataaagagTtggttgtagttctcatcaggaaccgtgggcaagggactgtgcgt |
| | | tcctcagcactcgaagctcttccgtgagaccTtgcccgcagggtgctctggttct |
| | | ttggggTtgctgtgctgtggcttcggaatTtgagcgtcttcccaccctccctccc |
| | | ctcccttcgccagcgttctgtctacaagaaagaataggcaggtgtccttggatat |
| | | CgtagttgctaatCgcctatacactgttctattacacctttctgctaaggatagg |
| | | gtttttggttttggttttggttttgttccccaccctccagtttggtttagttttg |
| | | gttttggcatttagggttttttgggggggagtaatatcttgtggtaaagacccat |
| | | Ctgacccaagataccttttttctcatactggaaccctaggcagcagttgctattt |
| | | ccctgagttagcaatagttttacagtattttgaggccttttgtccataattctca |
| | | cggaatCcctcagggatCagattagctgctgttgggatCaggaaattgggttaca |
| | | ccgctgaaatctcttgctggggcccttgttttgaattggaaagtcaggaggctgg |
| | | aacgaaggctcacaagttaacagtgccagctgctcttccagaagccctggattca |
| | | gtcccaccaatccatCgcgggtcacaaccatctgtaacttcagtcccaaggggtc |
| | | cgaAgccctcttctggctttgccctattattttatttatcttatCtgtttttgtc |
| | | ttgtcatCtggcaagcccagggggccattgggtgcaacttataaactgacttctg |
| | | tatCttaagaagccaaccatacagtgcttacattccagaaaaaaaatCtgccact |
| | | ttaacagcactagaactagggtttagagaagtatCataaaggtcaaatatCtttg |
| | | accaatatcaccagcaacctaaagctgttaagaaatctttgggccccagcttgac |
| | | ccaaggatacagtatcctagggaagttaccaaaatcagagatagtatgcagcagc |
| | | caggggtctcatgtgtggcactcaagctcacctatactcactactgtgcagacag |
| | | ctgtgttctctgtaatacttacatatttgtttaatacttcagggaggaaaagtca |
| | | gaagaccaggatctccagggcctcaACCGGTGGCCCaggCGGCCACCATGTGGAC |
| | | CCTGGTGAGCTGGGTGGCCTTAACAGCAGGGCTGGTGGCTGGAACGCGGTGCCCA |
| | | GATGGTCAGTTCTGCCCTGTGGCCTGCTGCCTGGACCCCGGAGGAGCCAGCTACA |
| | | GCTGCTGCCGTCCCCTTCTGGACAAATGGCCCACAACACTGAGCAGGCATCTGGG |
| | | TGGCCCCTGCCAGGTTGATGCCCACTGCTCTGCCGGCCACTCCTGCATCTTTACC |
| | | GTCTCAGGGACTTCCAGTTGCTGCCCCTTCCCAGAGGCCGTGGCATGCGGGGATG |
| | | GCCATCACTGCTGCCCACGGGGCTTCCACTGCAGTGCAGACGGGCGATCCTGCTT |
| | | CCAAAGATCAGGTAACAACTCCGTGGGTGCCATCCAGTGCCCTGATAGTCAGTTC |
| | | GAATGCCCGGACTTCTCCACGTGCTGTGTTATGGTCGATGGCTCCTGGGGGTGCT |
| | | GCCCCATGCCCCAGGCTTCCTGCTGTGAAGACAGGGTGCACTGCTGTCCGCACGG |
| | | TGCCTTCTGCGACCTGGTTCACACCCGCTGCATCACACCCACGGGCACCCACCCC |
| | | CTGGCAAAGAAGCTCCCTGCCCAGAGGACTAACAGGGCAGTGGCCTTGTCCAGCT |
| | | CGGTCATGTGTCCGGACGCACGGTCCCGGTGCCCTGATGGTTCTACCTGCTGTGA |
| | | GCTGCCCAGTGGGAAGTATGGCTGCTGCCCAATGCCCAACGCCACCTGCTGCTCC |
| | | GATCACCTGCACTGCTGCCCCCAAGACACTGTGTGTGACCTGATCCAGAGTAAGT |
| | | GCCTCTCCAAGGAGAACGCTACCACGGACCTCCTCACTAAGCTGCCTGCGCACAC |
| | | AGTGGGGGATGTGAAATGTGACATGGAGGTGAGCTGCCCAGATGGCTATACCTGC |
| | | TGCCGTCTACAGTCGGGGGCCTGGGGCTGCTGCCCTTTTACCCAGGCTGTGTGCT |
| | | GTGAGGACCACATACACTGCTGTCCCGCGGGGTTTACGTGTGACACGCAGAAGGG |
| | | TACCTGTGAACAGGGGCCCCACCAGGTGCCCTGGATGGAGAAGGCCCCAGCTCAC |
| | | CTCAGCCTGCCAGACCCACAAGCCTTGAAGAGAGATGTCCCCTGTGATAATGTCA |
| | | GCAGCTGTCCCTCCTCCGATACCTGCTGCCAACTCACGTCTGGGGAGTGGGGCTG |
| | | CTGTCCAATCCCAGAGGCTGTCTGCTGCTCGGACCACCAGCACTGCTGCCCCCAG |
| | | GGCTACACGTGTGTAGCTGAGGGGCAGTGTCAGCGAGGAAGCGAGATCGTGGCTG |
| | | GACTGGAGAAGATGCCTGCCCGCCGGGCTTCCTTATCCCACCCCAGAGACATCGG |
| | | CTGTGACCAGCACACCAGCTGCCCGGTGGGGCAGACCTGCTGCCCGAGCCTGGGT |
| | | GGGAGCTGGGCCTGCTGCCAGTTGCCCCATGCTGTGTGCTGCGAGGATCGCCAGC |
| | | ACTGCTGCCCGGCTGGCTACACCTGCAACGTGAAGGCTCGATCCTGCGAGAAGGA |
| | | AGTGGTCTCTGCCCAGCCTGCCACCTTCCTGGCCCGTAGCCCTCACGTGGGTGTG |
| | | AAGGACGTGGAGTGTGGGGAAGGACACTTCTGCCATGATAACCAGACCTGCTGCC |
| | | GAGACAACCGACAGGGCTGGGCCTGCTGTCCCTACCGCCAGGGCGTCTGTTGTGC |
| | | TGATCGGCGCCACTGCTGTCCTGCTGGCTTCCGCTGCGCAGCCAGGGGTACCAAG |
| | | TGTTTGCGCAGGGAGGCCCCGCGCTGGGACGCCCCTTTGAGGGACCCAGCCTTGA |
| | | GACAGCTGCTGTGAGGCCAggcCGGCCGaattcGATCCAGACATGATAAGATACA |
| | | TTGATGAGTTTGGACAAACCACAACTAGAATGCAGTGAAAAAAATGCTTTATTTG |
| | | TGAAATTTGTGATGCTATTGCTTTATTTGTAACCATTATAAGCTGCAATAAACAA |
| | | GTTAACAACAACAATTGCATTCATTTTATGTTTCAGGTTCAGGGGGAGGTGTGGG |
| | | AGGTTTTTTAGGGATCCTCAGgttaatcattaactacaaggaacccctagtgatg |
| | | gagttggccactccctctctgcgcgctcgctcgctcactgaggccgggcgaccaa |
| | | aggtcgcccgacgcccgggctttgcccgggcggcctcagtgagcgagcgagcgcg |
| | | c |

### EXAMPLES

### Material

rAAV particles comprising AAVtt were used. The heterologous nucleic acid (of SEQ ID NO.18) packaged into these particles comprised a granulin (PGRN) gene as a transgene. A routine triple transfection-method of a mammalian cell line was used to produce the particles.

Twenty-four male cynomolgus monkey (≥2-year-old, originating from Mauritius), were used for the study. Particles stocks were provided in 25 μL aliquots to be thawed, combined if needed (depending on the total volume to be administered) and dispensed on each dosing. Each aliquot was prepared and used on the day of dosing.

### Methods

### Intra-Cerebroventricular (ICV) Administrations

Intra-cerebroventricular (ICV) administrations were performed as follows: cerebrospinal fluid (CSF) was collected as a control/baseline before the start of the study. Similarly, Magnetic Resonance Imaging (MRI) were performed before the start of the study. At the start of the study, an incision was made, and the skin reflected, and a single hole was drilled through the skull over the target location (left hemisphere lateral ventricle). 22-gauge needles were used. Placement of the needle in the ventricle was verified using contrast media injection and fluoroscopy. The test material was then administered into the lateral ventricle at a rate 0.1 mL/min for a total dosing time dependent on volume. The needle remained in place for 2 minutes after the completion of the administration, and then retracted. Following completion of dosing, the skin was closed in a standard manner and the animals were allowed to recover. The animals were monitored for 62 days after surgery.

### Intra-Putaminal Administrations

Intra-putaminal administrations were performed as follows: Magnetic Resonance Imaging (MRI) were performed before the start of the study as a control/baseline. At the start of the study, an incision was made, and the skin reflected. Holes were drilled through the skull over the target locations. The syringe were primed 3 times to ensure the barrel moves freely. The administration was initiated at 1 μL/min as the device was lowered into place (~1 min to depth).

Once at depth, the test material was administered at a rate of 2 μL per minute for 5 minutes, then the rate was increased at 1 μL per minute until reaching a rate of 5 μL/min. The 5 μL/min flow rate was then maintained for the remainder of the administration. The cannulas remained in place for 10 minutes after the completion of the administration and then retracted and removed. The skin was closed in a standard manner and the animals were allowed to recover. The animals were monitored for 62 days after surgery.

### Histology and Microscopic Evaluation

Material for histology and microscopic evaluation were fixed by immersion for 72 to 96 hours at room temperature in methanol-free 4% paraformaldehyde (PFA) in 1× Phosphate Buffer Saline (PBS), then transferred to 1×PBS and kept at 2 to 8 °C until use. Representative slides were stained for PGRN (either for monkey PGRN only (see FIG. 1A) or for both human PGRN+monkey PGRN (see FIGS. 1B and 1C) with reagents generated in house) using standard protocols and procedures.

### Example 1—Biodistribution Study in Non-Human Primate

The objective of this study was to evaluate the biodistribution of an AAVTT-mediated gene therapy product when administered via intracerebroventricular (ICV) or Intra-putamina routes in cynomolgus monkeys and to investigate the suitability of various route of administration and dose combinations to reach appropriate brain coverage for neurodegenerative disease indications.

The animals were administered with a test material selected from either AAV-TT based particles comprising progranulin as a transgene or a vehicle (PBS with 0.001% pluronic F68). The following administrations were performed: intracerebroventricular (ICV)—unilateral administration (group 1) and intraparenchymal (IP)—Pre- and post-commissural, bilateral administration (groups 2 and 3). The experimental design can be found in Table 1.

TABLE 1

| | | | | |
|---|---|---|---|---|
| | | Number of | | |
| Group | Material | administrations | Total dose | No. animals |

intracerebroventricular (ICV) route

| | | | | |
|---|---|---|---|---|
| 1 | AAVTT + PGRN | 1 | 1e13 | 3 |

Intraputaminal (IP) route

| | | | | |
|---|---|---|---|---|
| 2 | AAVTT + PGRN | 2/hemisphere | 7.9e12 | 3 |
| 3 | Vehicle | 2/hemisphere | NA | 3 |

As shown in FIG. 1B, the intra putaminal administration allows a clear and widespread distribution of PGRN across cortical and subcortical regions, when the particles were administered at a dose of about 7.9e12 vg. To the contrary, no staining was observed with the vehicle (FIG. 1A). Only minimal expression of PGRN was observed when the animals were administered intracerebroventricularly at a dose of 1e13vg (higher dose than via intra-putaminal mute) as shown in FIG. 1C. The sole exception was where the needle tract was located (as shown by the arrow in FIG. 1C).

These results surprisingly showed that recombinant adeno-associated virus (rAAV) particles comprising AAV true type (AAVTT) capsids were able, when administered to the putamen, to spread from the putamen to the central nervous system (CNS) and to express the PGRN-transgene comprised in said particle all across the CNS (in particular to cortical and subcortical regions) of the primate in need of treatment. In particular, the administration to the putamen of rAAV particles according to the invention was superior to an intracerebroventricular (ICV) administration of the same rAAV particle in said primate (FIG. 1). These results support recombinant adeno-associated viral (rAAV) particle for use in the treatment of a neurodegenerative disease in a primate (a non human primate as well as a human), wherein the rAAV particle comprises: a) an AAV true type (AAVTT) capsid comprising or consisting of SEQ ID NO: 1 and b) a heterologous nucleic acid packaged therein, wherein the heterologous nucleic acid comprises: i) a 5′ inverted terminal repeat (ITR) or a fragment thereof and a 3′ ITR or a fragment thereof; ii) a transgene; and iii) one or more regulatory sequences that direct expression of said transgene; wherein the rAAV particle is administered to the putamen of said primate, and wherein the transgene encodes a secreted lysosomal protein, preferably human progranulin (PGRN), an active fragment thereof and/or an active variant thereof.

### REFERENCES

Ojala D. S. et al., 2015, The Neuroscientis, 21(1): 84-98
Bartlett J. S. et al., 1998, Hum. Gene Ther., 9(8):1181-6
Mandel R. J. et al., 2004, Curr. Opin. Mol. Ther.: 6(5):482-90
Wood et al. 2022, Front Mol Biosci, 9:965089
Coyle-Gilchrist I. T. et al., 2016, Neurology, 86(18):1736-43
Rohrer J D. et al., 2009, Neurology, 73(18):1461-6
Le Ber I., et al., 2008, Brain, 131(3):732-46
Kamate M. et al., 2019, Brain, 41(6):542-545
Irwin D. et al., 2009, J. Neurol. Sci., 276(1-2): 9-13
Wozar F. et al., 2022, Kin. Mobi. Augenheilkd., 239(3):270-74
Pignataro D. et al., 2018, J. Neural. Transm., 125:575-589
FDA draft guidance for industry “Human Gene Therapy for Neurodegenerative Diseases”, 2022 WO02015121501
Tordo J. et al., 2018, Brain, 141:2014-31
O'Leary C. et al., 2016, Molecular Genetics and Metabolism, 120:S254

1. A method of treating a neurodegenerative disease in a primate by administering to the primate a therapeutic amount of a recombinant adeno-associated viral (rAAV) particle, wherein the rAAV particle comprises:
a) an AAV true type (AAVTT) capsid comprising or consisting of SEQ ID NO: 1 and
b) a heterologous nucleic acid packaged therein; wherein the heterologous nucleic acid comprises:
i) a 5′ inverted terminal repeat (ITR) or a fragment thereof and a 3′ ITR or a fragment thereof;
ii) a transgene; and
iii) one or more regulatory sequences that direct, assist and/or control expression of said transgene;
wherein the treatment comprises the administration of said rAAV particle to the putamen of said mammal, and wherein the transgene encodes a secreted lysosomal protein, preferably human progranulin (PGRN), an active fragment thereof and/or an active variant thereof.

2. A method of delivering to the central nervous system of a primate a therapeutic amount of a recombinant adeno-associated viral (rAAV) particle, wherein the rAAV particle comprises:
a) an AAV true type capsid comprising or consisting of SEQ ID NO: 1 and
b) a heterologous nucleic acid packaged therein; wherein the heterologous nucleic acid comprises:
i) a 5′ inverted terminal repeat (ITR) or a fragment thereof and a 3′ ITR or a fragment thereof;
ii) a transgene; and
iii) one or more regulatory sequences that direct, assist and/or control expression of said transgene;
wherein the method comprises delivering the rAAV particle to the putamen of said primate, and
wherein the transgene encodes a secreted lysosomal protein, preferably human progranulin (PGRN), an active fragment thereof and/or an active variant thereof.

3. A recombinant adeno-associated virus (rAAV) particle for use in the treatment of a neurodegenerative disease in a primate, wherein the rAAV particle comprises:
a) an AAV true type (AAVTT) capsid comprising or consisting of SEQ ID NO: 1 and
b) a heterologous nucleic acid packaged therein; wherein the heterologous nucleic acid comprises:
i) a 5′ inverted terminal repeat (ITR) or a fragment thereof and a 3′ ITR or a fragment thereof;
ii) a transgene; and
iii) one or more regulatory sequences that direct, assist and/or control expression of said transgene;
wherein the rAAV particle is administered to the putamen of said primate, and wherein the transgene encodes a secreted lysosomal protein, preferably human progranulin (PGRN), an active fragment thereof and/or an active variant thereof.

4. A recombinant adeno-associated virus (rAAV) particle for use in the delivery of a heterologous nucleic acid to the central nervous system of a primate, wherein the rAAV particle comprises:
a) an AAV true type (AAVTT) capsid comprising or consisting of SEQ ID NO: 1 and
b) a heterologous nucleic acid packaged therein; wherein the heterologous nucleic acid comprises:
i) a 5′ inverted terminal repeat (ITR) or a fragment thereof and a 3′ ITR or a fragment thereof;
ii) a transgene; and
iii) one or more regulatory sequences that direct, assist and/or control expression of said transgene;
wherein the rAAV particle is administered to the putamen of said primate, and wherein the transgene encodes a secreted lysosomal protein, preferably human progranulin (PGRN), an active fragment thereof and/or an active variant thereof.

5. A pharmaceutical composition comprising a recombinant adeno-associated viral (rAAV) particle and one or more carriers and/or excipients, wherein the rAAV particle comprises:
a) an AAV true type capsid comprising or consisting of SEQ ID NO: 1 and
b) a heterologous nucleic acid packaged therein; wherein the heterologous nucleic acid comprises:
i. a 5′ inverted terminal repeat (ITR) or a fragment thereof and a 3′ ITR or a fragment thereof;
ii. a transgene; and
iii. one or more regulatory sequences that direct, assist and/or control expression of said transgene;
wherein the pharmaceutical composition is administered to the putamen of a primate in need thereof, and wherein the transgene encodes a secreted lysosomal protein, preferably human progranulin (PGRN), an active fragment thereof and/or an active variant thereof.

6. The pharmaceutical composition according to claim 5, wherein said composition is for use in the treatment of a neurodegenerative disease in a primate.

7. The method, the rAAV particle for use or the pharmaceutical composition according to any one of the preceding claims, wherein the human progranulin (PGRN), active fragment thereof and/or active variant thereof: 1) has an amino acid sequence comprising SEQ ID NO: 4 or a sequence having at least 95%, at least 96%, at least 97%, at least 98% or at least 99% sequence identity thereto or 2) is encoded by a nucleic sequence encoding PGRN comprises SEQ ID NO: 7 or a sequence having at least 95%, at least 96%, at least 97%, at least 98% or at least 99% sequence identity thereto.

8. The method, the rAAV particle for use or the pharmaceutical composition according to any one of the preceding claims, wherein the neurodegenerative disease is neuronal ceroid lipofuscinosis such as NCL type 11, mucopolysaccharidosis (MPS), progressive supranuclear palsy (PSP), corticobasal syndrome, Parkinson's disease, dementia with Lewy bodies, Alzheimer's disease or amyotrophic lateral sclerosis with or without dementia (ALS or ALS-D).

9. The method, the rAAV particle for use or the pharmaceutical composition according to any one of claim 1 to 7, wherein the neurodegenerative disease is an adult-onset neurodegenerative disease.

10. The method, the rAAV particle for use or the pharmaceutical composition according to claim 9, wherein the disease is fronto-temporal dementia (FTD), NCL type 11 and/or PGRN haploinsufficiency.

11. The method, the rAAV particle for use or the pharmaceutical composition according to anyone of the preceding claims wherein the rAAV particle comprising AAVTT capsid spreads from the putamen to cortical and subcortical regions.

12. The method, the rAAV particle for use or the pharmaceutical composition according to anyone of the preceding claims wherein the administration comprises a total dose comprising at least 1×10⁶ viral genome (vg) of rAAV particle.

13. The method, the rAAV particle for use or the pharmaceutical composition according to any one of the preceding claims wherein the administration to the putamen of the rAAV particle comprising AAVTT capsid is superior to an intracerebroventricular (ICV) administration of the rAAV particle in said primate.

14. The method, the rAAV particle for use or the pharmaceutical composition according to any one of the preceding claims wherein the administration of the total dose comprises at least two doses, preferably administered as one dose administered in the putamen of the right hemisphere and one dose administered in the putamen of the left hemisphere.

15. The method, the rAAV particle for use or the pharmaceutical composition according to any one of the preceding claims wherein:
a) the 5′ ITR comprises or consisting of:
) SEQ ID NO: 2, or a sequence having at least 95%, at least 96%, at least 97%, at least 98% or at least 99% sequence identity thereto; or
ii) SEQ ID NO: 5 or a sequence having at least 95%, at least 96%, at least 97%, at least 98% or at least 99% sequence identity thereto; and
b) the 3′ ITR comprises or consisting of:
) SEQ ID NO: 3, or a sequence having at least 95%, at least 96%, at least 97%, at least 98% or at least 99% sequence identity thereto, or
ii) SEQ ID NO: 6 or a sequence having at least 95%, at least 96%, at least 97%, at least 98% or at least 99% sequence identity thereto.

16. The method, the rAAV particle for use or the pharmaceutical composition according to any one of the preceding claims wherein the one or more regulatory sequences that direct, assist and/or control expression of said transgene is selected from the groups consisting of:
a. one or more transcription initiation sequences (such as a promoter),
b. one or more translation initiation sequences,
c. one or more mRNA stability sequences,
d. one or more polyadenylation sequences,
e. one or more secretory sequences,
f. one or more enhancer sequences,
g. one or more introns,
h. one or more TATA boxes,
i. one or more microRNA targeted sequences,
j. one or more polylinker sequences facilitating the insertion of a DNA fragment within a vector,
k. one or more splicing signal sequences,
l. one or more transcription termination sequences (such as polyadenylation sequences) or
m. any combinations of any one or more of the groups a. to l.


SEQUENCE LISTING (18 sequences)

SEQ ID NO: 1 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 2 - DNA, 128 bp, synthetic construct
  source 1..128: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 128 bp, synthetic construct
  source 1..128: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 4 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 5 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 6 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 7 - DNA, 1782 bp, synthetic construct
  source 1..1782: mol_type = other DNA
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 8 - DNA, 229 bp, synthetic construct
  source 1..229: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 9 - DNA, 2000 bp, synthetic construct
  source 1..2000: mol_type = other DNA
gcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctca

SEQ ID NO: 10 - DNA, 2235 bp, synthetic construct
  source 1..2235: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctca

SEQ ID NO: 11 - DNA, 125 bp, synthetic construct
  source 1..125: mol_type = other DNA
gcgctccctcctctcggagagagggctgtggtaaaacccgtccggaaattggccgccgctgccgccaccgccgccgccgccgccgcgccgagcggaggaggaggaggaggcgaggaggagagact

SEQ ID NO: 12 - DNA, 875 bp, synthetic construct
  source 1..875: mol_type = other DNA
gtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcg

SEQ ID NO: 13 - DNA, 962 bp, synthetic construct
  source 1..962: mol_type = other DNA
ttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcag

SEQ ID NO: 14 - DNA, 38 bp, synthetic construct
  source 1..38: mol_type = other DNA
ggaggaaaagtcagaagaccaggatctccagggcctca

SEQ ID NO: 15 - DNA, 2337 bp, synthetic construct
  source 1..2337: mol_type = other DNA
ctctaccattacgttttatcctcagactctatctccccattttaaaggaatattatttttaaatgctacactctcattttttaaatggctccttttaattctactgctaaaatacttttggtacaatatgcctttttttctattttttttttttagtgcaagtataaaatatgtcatttaaatctcttttatctaatttaaggaacttaagattttcttcccaaaatttcacaaggtaggaaaatgatgtactttatttttgtgaatgatattgcactgtagatcttgctgtttcttgctttgttctcaattaaatatcatgtttcctctacagctttatagacattttgatcagattaaggacattctaattatagttctttaaggtgtttttaaaatcatatgtaggtattgaattttattaaatgctttttcctgcatgtattgggatactcacatgatctgttcttaaaattataattgatttctaattttaaaccatccttgcattcgtggaataaaactcagcctgaaggtgtggctggagagatggcttgttgctcttgcagaggacccaagttcagatcctattctctgtatatgcaaatacctgtattctcacaccccaacatacacacacacacacacacacacacacacacacactaccactcttacccacttgttttttacatttctatcttagtattttatgtgtataagtgttttgcctggtgctcactgtggtcagaagagggcacaggattccctgagactagaattacaaatggtttagaatggccatatgggaaatcctctagatttcccccactgtagtaagatattcacttaggtgatcctgtcccaaagtcagccatcatccttattttttttctttctttccatctgatatccaatacttttggctcaatttttaacataaatcttaactatcacaacttatcagatttcaactgctactgtcctggttaaagccttcatcatctatctttcttcaactgctgccaggacctctggaccagccagttcttcattcttcactggcaacataggttttatggtgacagctagtgactcaaatatttatcaagggcttctcatctcaaaataatctcctagttcttttggtggcctaggtctctctccagtcacactggcctccttagtaaggcaggcatagtccttccttagagtgtttaaacttgcctagaatgttttccccaattacccatattgggagacgacatgagggcaaaagctagagggtatcataatagcacttcttttgtccttgccctatctatttcaaagtctttatctctgtgcaaaattttaagttctactttcttgtatgtttagtatgactcttccttaccaggagtctagtttgtctccttgttcagtactaaaacagtgcctagcaaataaatgaatagagaggggagccaaatttgaatcagaaagtctcttgttgcatagtgtttaaaaaacaaacaaagaaagaaagtctcttgttgagcatttgtttagcacaaagagcattggatgctgactggtatcagggtaaggctgctttgacaatgctccctctggcctcactcccttttatacgtacttccatcaaaccatctgattcaacaatgacagaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcttaattaaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 16 - DNA, 2108 bp, synthetic construct
  source 1..2108: mol_type = other DNA
ctctaccattacgttttatcctcagactctatctccccattttaaaggaatattatttttaaatgctacactctcattttttaaatggctccttttaattctactgctaaaatacttttggtacaatatgcctttttttctattttttttttttagtgcaagtataaaatatgtcatttaaatctcttttatctaatttaaggaacttaagattttcttcccaaaatttcacaaggtaggaaaatgatgtactttatttttgtgaatgatattgcactgtagatcttgctgtttcttgctttgttctcaattaaatatcatgtttcctctacagctttatagacattttgatcagattaaggacattctaattatagttctttaaggtgtttttaaaatcatatgtaggtattgaattttattaaatgctttttcctgcatgtattgggatactcacatgatctgttcttaaaattataattgatttctaattttaaaccatccttgcattcgtggaataaaactcagcctgaaggtgtggctggagagatggcttgttgctcttgcagaggacccaagttcagatcctattctctgtatatgcaaatacctgtattctcacaccccaacatacacacacacacacacacacacacacacacacactaccactcttacccacttgttttttacatttctatcttagtattttatgtgtataagtgttttgcctggtgctcactgtggtcagaagagggcacaggattccctgagactagaattacaaatggtttagaatggccatatgggaaatcctctagatttcccccactgtagtaagatattcacttaggtgatcctgtcccaaagtcagccatcatccttattttttttctttctttccatctgatatccaatacttttggctcaatttttaacataaatcttaactatcacaacttatcagatttcaactgctactgtcctggttaaagccttcatcatctatctttcttcaactgctgccaggacctctggaccagccagttcttcattcttcactggcaacataggttttatggtgacagctagtgactcaaatatttatcaagggcttctcatctcaaaataatctcctagttcttttggtggcctaggtctctctccagtcacactggcctccttagtaaggcaggcatagtccttccttagagtgtttaaacttgcctagaatgttttccccaattacccatattgggagacgacatgagggcaaaagctagagggtatcataatagcacttcttttgtccttgccctatctatttcaaagtctttatctctgtgcaaaattttaagttctactttcttgtatgtttagtatgactcttccttaccaggagtctagtttgtctccttgttcagtactaaaacagtgcctagcaaataaatgaatagagaggggagccaaatttgaatcagaaagtctcttgttgcatagtgtttaaaaaacaaacaaagaaagaaagtctcttgttgagcatttgtttagcacaaagagcattggatgctgactggtatcagggtaaggctgctttgacaatgctccctctggcctcactcccttttatacgtacttccatcaaaccatctgattcaacaatgacagaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcttaattaa

SEQ ID NO: 17 - DNA, 198 bp, synthetic construct
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttag

SEQ ID NO: 18 - DNA, 4566 bp, synthetic construct
  source 1..4566: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctctgctagcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctcaaccggtggcccaggcggccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaggccaggccggccgaattcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagggatcctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc